(19) United States
(12) Patent Application Publication
(10) Pub. No.: US 2026/0294304 A1
(43) Pub. Date: Oct. 1, 2026

(54) MONITORING SOCIABILITY OF A USER

(71) Applicant: Cochlear Limited, Macquarie University, NSW (AU)

(72) Inventors: Paul REINHART, Alexandria, VA (US); Bridget TIERNAN, Lone Tree, CO (US); Birgit PHILIPS, Mechelen (BE); Peter GIBSON, South Coogee (AU); Kerrie PLANT, Melbourne (AU)

(21) Appl. No.: 19/477,315

(22) PCT Filed: Apr. 24, 2024
(86) PCT No.: PCT/IB2024/054020

§ 371 Date: Oct. 21, 2025

Related U.S. Application Data
(60) Provisional application No. 63/499,314, filed on May 1, 2023.

(51) Int. Cl. A61B 5/16; A61B 5/00; A61N 1/36; G10L 25/78

(52) U.S. Cl. CPC A61B 5/167; A61B 5/4803; A61N 1/36038; G10L 25/78

(57) ABSTRACT

Presented herein are techniques for monitoring (e.g., detecting, determining, tracking, calculating, etc.) a sociability index of a user (e.g., a recipient) of a “user device,” that is a device that is carried by, worn by, or implanted in, the user. The sociability index can include, but are not limited to, a listening situation of the user and/or an activity associated with the user in the listening situation.

### BACKGROUND

### Technical Field

The present disclosure relates generally to monitoring sociability of a user of a device, such as hearing or medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: determining, via a processor, a first metric of a listening situation associated with a hearing device user; determining, via the processor, a second metric of an activity associated with the hearing device user in the listening situation; and determining, via the processor, a sociability index based on the first metric and the second metric. The processor can be implemented, for example, as part of a cochlear implant system, a mobile device, computing device, and/or a network platform (e.g., a cloud server).

In another aspect, one or more non-transitory computer readable storage media are provided. The one or more non-transitory computer readable storage media comprise instructions that, when executed by a processor, cause the processor to: receive data from a sensor associated with a device configured to be worn by a user; determine, based on the data, a listening situation of the user and an activity of the user in the listening situation; and determine a sociability index based on the listening situation of the user and the activity of the user in the listening situation.

In another aspect, a method is provided. The method comprises: receiving, via a processor, biometric data of a hearing device user; determining, via the processor, a listening effort metric based on the biometric data; and determining, via the processor, a sociability index based on the listening effort metric.

In another aspect, a device is provided. The device comprises: a memory configured to store instructions; and one or more processors that, upon executing the instructions stored on the memory, are configured to: determine a person in conversational engagement with a user of the device; determine a social network metric based on the person in conversational engagement with the user; and determine a sociability index based on the social network metrics.

### BRIEF DESCRIPTION OF THE DRAWINGS

Embodiments of the present disclosure are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is a diagram illustrating various metrics used to determine a sociability index, in accordance with certain embodiments presented herein;

FIG. 3 is a flowchart illustrating a method for changing how a sociability index is determined, in accordance with certain embodiments presented herein;

FIG. 4 is a schematic diagram illustrating a mobile device configured to display information regarding a sociability index, in accordance with certain embodiments presented herein;

FIG. 5 is a schematic diagram illustrating a network of systems that communicate information regarding a sociability index, in accordance with certain embodiments presented herein;

FIG. 6A is a flowchart illustrating a method for determining a sociability index, in accordance with certain embodiments presented herein;

FIG. 6B is a flowchart illustrating a method for determining a sociability index, in accordance with certain embodiments presented herein;

FIG. 7 is a flowchart illustrating a method for providing a recommendation based on a sociability index, in accordance with certain embodiments presented herein;

FIG. 8 is a schematic diagram illustrating a vestibular stimulator system with which aspects of the techniques presented herein can be implemented; and

FIG. 9 is a schematic diagram illustrating a retinal prosthesis system with which aspects of the techniques presented herein can be implemented.

### DETAILED DESCRIPTION

Presented herein are techniques for monitoring (e.g., detecting, determining, tracking, calculating, etc.) a sociability index of a user (e.g., a recipient) of a “user device,” that is a device that is carried by, worn by, or implanted in, the user. The sociability index can include, but are not limited to, a listening situation of the user and/or an activity associated with the user in the listening situation.

A user device (e.g., a portable device carried by a user, a wearable device worn by a user, an implantable device having one or more components implanted in the user, etc.) is configured to receive information that can be used to monitor the sociability index of the user. The received information can include, for example, acoustic data associated with the user and/or a surrounding environment, biometric data of the user, and/or location data of the user. Such information can be received by sensor data and/or user inputs. For example, in embodiments in which the user device is a hearing device, such as a cochlear implant or a hearing aid, a microphone or acoustic detector can detect the acoustic data.

In certain examples, the techniques presented herein can be beneficial for monitoring the sociability index of a user to determine whether the user is functioning desirably in social settings. For instance, individuals utilizing a hearing device to mitigate their hearing impairment can have difficulty maintaining a desired sociability because of different factors, including continued difficulty in hearing, difficulty discerning competing noise sources (e.g., other speakers), and unfamiliarity with usage of their hearing device. An individual's health and wellness can be affected by undesirable or low social activity. Therefore, it is desirable to maintain or improve an individual's social patterns to establish greater wellbeing for the individual. The sociability index provides a more tangible representation regarding whether the user's social patterns are desirable. For example, providing the user with information related to the sociability index can prompt the user to adjust, maintain, or otherwise manage their behavior to achieve a target sociability index. Additionally or alternatively, the sociability index can be compared to a target or threshold level, and additional information or assistance can be provided based on the comparison, such as in response to the sociability index being below the target level. As an example, a recommendation can be provided to the user based on their sociability index. As another example, another individual (e.g., a significant other, a caretaker, a healthcare provider, a therapist, etc.) can be notified to provide assistance to the user. Thus, the monitoring of the sociability index, as well as the additional actions performed based on the sociability index, can help fulfill the user's social goals and improve their comfort to utilize the user device.

There are a number of different types of user devices in/with which embodiments of the present disclosure can be implemented. Merely for ease of description and illustration, the techniques presented herein are primarily described with reference to a specific user device in the form of a cochlear implant system.

There are a number of different types of electronic devices in/with which the techniques presented herein can be implemented. Merely for ease of description, the techniques presented herein are primarily described with reference to a specific electronic device in the form of a cochlear implant system. However, it is to be appreciated that the techniques presented herein can also be partially or fully implemented by any of a number of different types of user devices, including consumer electronic devices (e.g., mobile devices, wearables, computing devices, televisions, appliances/white goods, etc.), other medical devices, diagnostic equipment, etc. For example, the techniques presented herein could be implemented by hearing devices, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. In further embodiments, the techniques presented herein can be implemented with air purifiers or air sensors (e.g., automatically adjust depending on environment), hospital beds, identification (ID) badges/bands, or other hospital equipment or instruments, or the like.

As used herein, the term “hearing device” is to be broadly construed as any device that delivers sound signals to a user in any form, including in the form of acoustical stimulation, mechanical stimulation, electrical stimulation, optical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones and other listening devices).

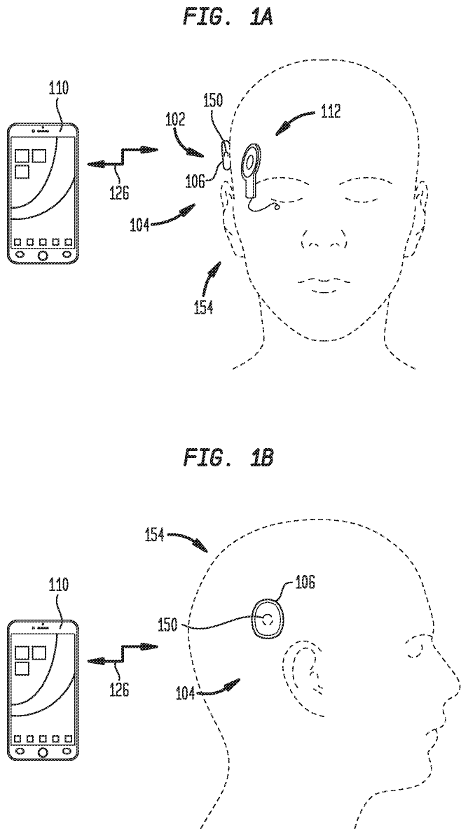

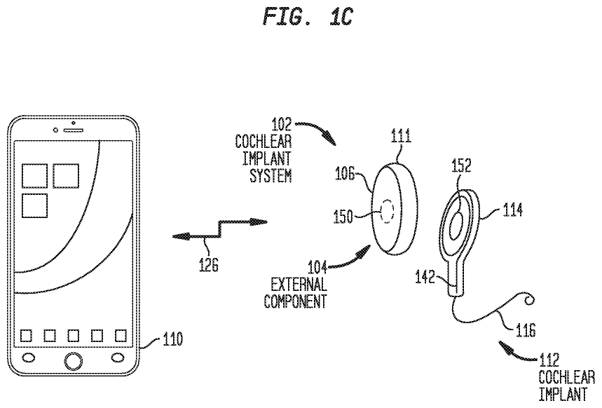

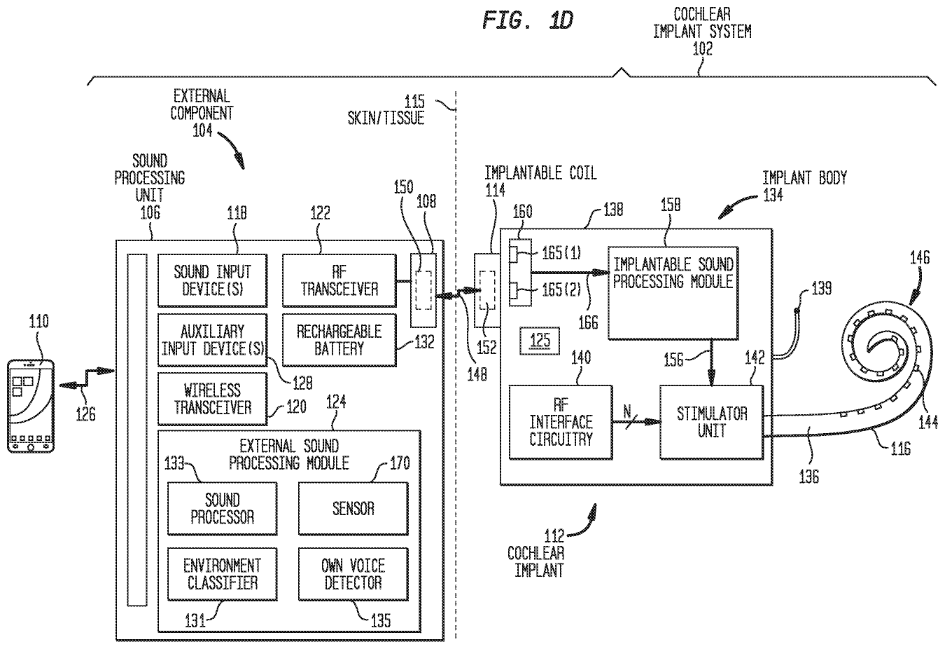

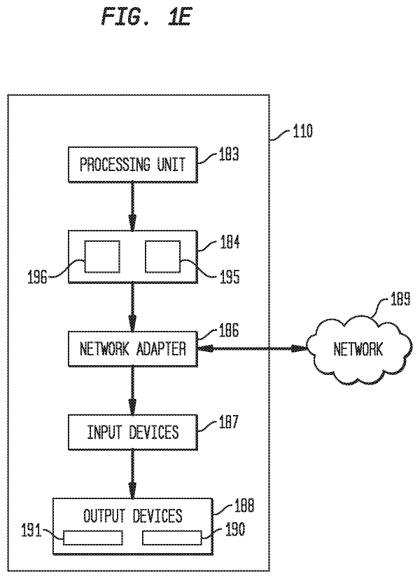

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 may comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals, which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. IE, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on, or near the sound processing unit 106. However, it is to be appreciated that one or more input devices may include additional types of input devices and/or fewer input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations which are represented in FIG. 1D by an environmental classifier 131, a sound processor 133, and an own voice detector 135. Each of the environmental classifier 131, the sound processor 133, and the own voice detector 135 can be formed by one or more processors (e.g., one or more Digital Signal Processors (DSPs), one or more uC cores, etc.), firmware, software, etc. arranged to perform operations described herein. That is, the environmental classifier 131, the sound processor 133, and the own voice detector 135 can each be implemented as firmware elements, partially or fully implemented with digital logic gates in one or more application-specific integrated circuits (ASICs), partially or fully in software, etc. Although FIG. 1D illustrates an environmental classifier 131, a sound processor 133, and an own voice detector 135 as being implemented/performed at the external sound processing module 124, it is to be appreciated that these elements (e.g., functional operations) could also or alternatively be implemented/performed as part of the implantable sound processing module 158, as part of the external device 110, etc.

The environmental classifier 131 (e.g., one or more processing elements implementing firmware, software, etc.) is configured to determine an environmental classification of the sound environment (i.e., determines the “class” or “category” of the sound environment) associated with the input audio signals received at the cochlear implant system 102. The environmental classifier 131 includes a decision tree, sometimes referred to as an environmental classifier decision tree that, in certain embodiments, can be trained/updated. The own voice detector 135 (e.g., one or more processing elements implementing firmware, software, etc.) is configured to perform an own voice detection (OVD) process associated with the input audio signals received at the cochlear implant system 102. The own voice detector 135 includes a decision tree, sometimes referred to herein as an OVD decision tree, that can be trained/updated. To provide the ability to train/update the OVD decision tree and/or the environmental classifier decision tree, the decision trees are stored in volatile memory and exposed to, for example, another process for updating thereof. As such, the environmental classifier 131 and the own voice detector 135 are at least partially implemented in volatile memory. The environmental classification decision tree and the own voice detection decision tree can be dynamically updated on/by the device itself (e.g., cochlear implant system 102), or updated using an external computing device (e.g., external device 110).

As used herein, OVD generally refers to a process in which speech signals received at a hearing device, such as a cochlear implant system 102, are classified as either including the “voice” or “speech” of the user (e.g., recipient) of the hearing device (referred to herein as the recipient's own voice or simply “own voice”) or a voice or speech generated by one or more persons other than the recipient (referred to herein as “external voice”).

Returning to the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.), RF interface circuitry 140, and a stimulator unit 142. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108, and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive, and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112, and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode, the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include an implantable sound processing module 158 and a plurality of implantable sound sensors 165(1), 165(2) that collectively form a sensor array 160. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

According to the techniques of the present disclosure, external sound processing module 124 may also include one or more other sensors 170 that determine other data. As an example, the sensors 170 include a location sensor (e.g., a global positioning system (GPS)) configured to determine a geographical location of the cochlear implant system 102 and therefore of the user associated with the cochlear implant system 102. As another example, the sensors 170 include a sensor, such as a heartbeat monitor, a temperature sensor, a blood pressure sensor, configured to determine various biometric data associated with the user. Such sensors 170 may be implemented in, for example, micro electromechanical systems (MEMS) or with other technology suitable for the particular application.

As also illustrated in FIG. 1D, in certain examples, an inertial measurement unit (IMU) 180 including one or more sensors 185 is incorporated into implantable sound processing module 158 of implant body 134. The IMU 180 is configured to measure the inertia of the user's head, that is, motion of the user's head. As such, IMU 180 comprises one or more sensors 185 each configured to sense one or more of rectilinear or rotatory motion in the same or different axes. Examples of sensors 185 that may be used as part of the IMU 180 include accelerometers, gyroscopes, inclinometers, compasses, and the like. As discussed herein, data received at the environment classifier 131, the sound processor 133, the own voice detector 135, the sensors 170, and/or the IMU 180 can be used to monitor the sociability index of a user of the cochlear implant system 102.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include Electronically-Erasable Programmable Read-Only Memory (EEPROM), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises sociability index logic 195 that, when executed, enables the processing unit 183 to perform aspects of the techniques presented. The memory 184 also further comprises sociability index data 196, which may include various data (e.g., previously determined sociability indexes) that is utilized by and/or updated by the sociability index logic 195.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

As noted herein, user devices, such as the cochlear implant system 102 of FIGS. 1A-1D, hearing devices, smartwatches, etc., can include microphones, location sensors, biometric sensors, or other sensors, which can obtain/capture data, such as acoustic data, location data, and/or biometric data, that can be utilized to monitor one or more aspects of a sociability index. For example, data related to a listening situation and data related to activity of a user in the listening situation are used to determine an overall sociability index. Indeed, such data indicate various aspects related to a user's effort and function in social settings to provide the sociability index that accurately represents the user's social patterns. In certain embodiments, the data is quantified and/or weighted, and the sociability index is calculated based on such data, such as using an equation, data model, or algorithm that defines a relationship between the sociability index and the data.

According to an aspect of the present disclosure, systems and methods are provided that automate a process for determining the sociability index and outputting a control signal based on the determined sociability index. In some example embodiments, the control signal can cause display of the sociability index, such as via a mobile device that informs the user of the sociability index. Providing the user with awareness of the sociability index can urge the user to monitor, adjust, and/or maintain certain behaviors to manage (e.g., increase) their sociability index. Additionally, the sociability index can be compared to a target or threshold level (e.g., a numerical value), and the control signal can be output based on the comparison, such as in response to a difference between the sociability index and the target level exceeding a threshold value. By way of example, a first control signal is output based on the sociability index being less than the target level, such as to provide a recommendation or other information that helps the user increase the sociability index. A second signal can be output based on the sociability index being greater than the target level, such as to notify the user that their sociability index is at a desirable level.

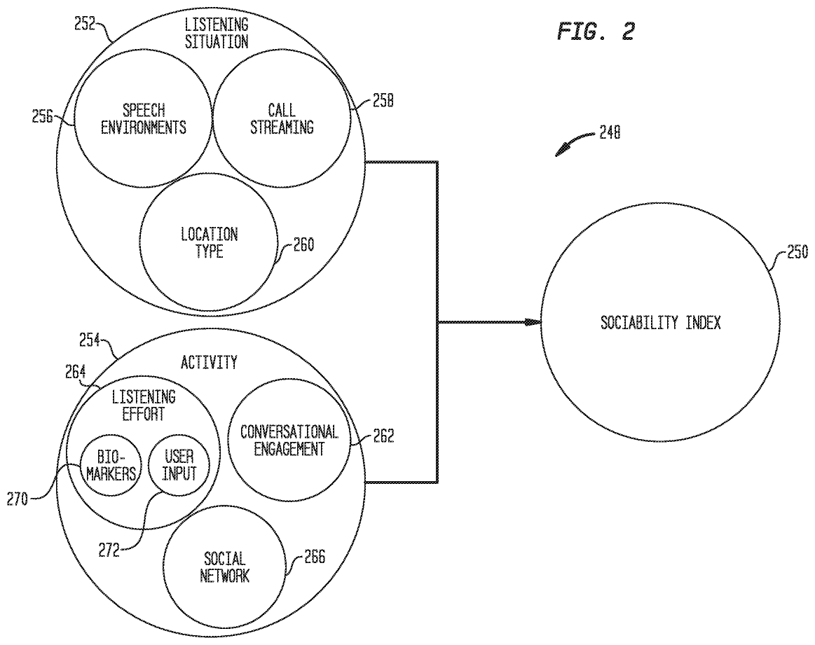

FIG. 2 is a diagram 248 illustrating different metrics that can be used to determine a sociability index 250. These metrics could, for example, be used by the system of FIGS. 1A-1E (or other devices/systems). The diagram 248 organizes the metrics into a first category 252 and a second category 254. The first category 252 includes a listening situation, and the second category 254 includes an activity in a particular listening situation. For example, the first category 252 indicates the context or setting of the user, such as the surroundings of the user. The first category 252 includes various sub-categories, including a speech environment sub-category 256, a call streaming sub-category 258, and a location type sub-category 260. The speech environment sub-category 256 indicates whether the user is in an environment in which the user is able to speak or converse (e.g., with another person). As an example, attributes of input audio signals (represented by electrical input signals) are evaluated/analyzed (e.g., by the environmental classifier 131). Based on the analysis, a “class” of the speech environment associated with the input audio signals is determined. Such categorization can include a number of predetermined speech environment classes, such as “speech in quiet,” “speech in noise,” “quiet,” “noise,” and “music,” although other categories are possible.

For instance, the speech environment class is determined by calculating, in real-time, time-varying features from the input audio signals and analyzing the calculated time-varying features using a type of decision tree (e.g., environmental classification decision tree) or other algorithm. The decision tree includes a number of hierarchical or linked branches/nodes that each perform evaluations, comparisons, or checks using at least one of the time-varying features to determine the sound environment classification at the branch ends (leaves). That is, the decision tree traverses its “branches” until it arrives at a “leaf” and decides “speech in quiet,” “speech in noise,” “quiet,” “noise,” or “music.”

The speech environment class of the speech environment sub-category 256 impacts the sociability index 250 based on determination of environments in which speech participation is available. For instance, increased determination of the “speech in quiet” or “speech in noise” sub-category (e.g., reduced determination of “quiet,” “noise,” or “music”) can increase the sociability index 250. In contrast, reduced determination of the “speech in quiet” or “speech in noise” (e.g., increased determination of “quiet,” “noise,” or “music”) can reduce the sociability index 250. Thus, the user is encouraged to participate in more settings in which speech participation is available to increase the sociability index 250.

The call streaming sub-category 258 indicates usage of electronic devices to converse with others. For example, the call streaming sub-category 258 encompasses phone calls, video calls, voice recording exchanges, and so forth. Indeed, call streaming can occur independently of the speech environment sub-category 256. In addition, call streaming can be performed with another person or with an automated system (e.g., an automated menu).

Data related to call streaming can be determined based on operation of an electronic device, such as a mobile phone, a laptop computer, a desktop computer, or a tablet, to effectuate the call streaming. By way of example, operation of the electronic device to communicatively couple to another electronic device can be determined to indicate call streaming. As another example, streamed calls can be distinguished from other audio streaming (e.g., music streaming, video streaming, etc.) based on firmware classification by the electronic device, usage of OVD to determine user vocalizations, and the like.

In some embodiments, a quantity of call streaming occurrences can be determined for the call streaming sub-category 258. Additionally or alternatively, a duration of time associated with streamed calls can be determined for the call streaming sub-category 258. Increased call streaming increases the sociability index 250 to encourage usage of an electronic device for conversational engagement.

The location type sub-category 260 indicates various types of environments or settings of the user. The location type can include broader classifications, such as a building or structure type (e.g., a residence, an office, a restaurant, a public venue, etc.) or a geographical location (e.g., a town, a state, etc.). The location type can also include more specific classifications, such as a particular event (e.g., a town meeting, a family gathering, a celebration, etc.) or an approximate number of other people in the location.

As an example, the location type can be determined based on location data, such as geographical coordinates of the user. As another example, the location data can be determined based on movement data (e.g., provided by the IMU 180). As a further example, the location type can be determined based on information that is stored on or retrieved from other sources. For instance, a particular event in which the user is participating can be determined based on a stored calendar or email invite, which corresponds to the determined user location and/or a determined time/date. The location type can therefore be determined based on various data, including sensor data and stored data.

A diverse array of location types can increase the sociability index 250 to encourage the user to attend potentially novel or unfamiliar settings, such as settings having different conversational environments. For example, a greater quantity of attended location types or an increased frequency of determined new locations types can increase the sociability index 250, whereas a fewer quantity of attended location types or an increased frequency of the same determined location type can reduce the sociability index 250. Thus, the location of the user is compared to previous locations of the user, and a difference between the location and the previous locations increases the sociability index 250. In addition, increased duration of time could be an increase in the index, similar to call streaming (e.g., not leaving the party early) The second category 254 indicates user activity and includes a conversational engagement sub-category 262, a conversational (e.g., listening, speaking) effort sub-category 264, and a social network sub-category 266. The conversational engagement sub-category 262 indicates the user's participation level in a conversation and can include a quantitative and qualitative indication of the user's participation. As an example, the conversational engagement sub-category 262 can include a duration of time during which the user speaks and/or a quantity of words or sentences spoken by the user (e.g., relative to that spoken by other people). As another example, the conversational engagement sub-category 262 can indicate whether the user's conversational content is contributory or substantive based on a quantity or proportion of certain spoken words, such as filler words (e.g., “um,” “uh,” etc.), interrogative words (e.g., “what,” “when,” etc.), pro-sentences (e.g., yes, no, etc.), and the like. Increased contributory conversational content increases the sociability index 250.

OVD is used (e.g., by the own voice detector 135) to evaluate the conversational engagement sub-category 262. Specifically, input audio signals are classified as being “own voice” (i.e., the hearing device recipient is speaking within the set of input audio signals) or as “external voice” (i.e., someone other than the hearing device recipient is speaking within the set of input audio signals). For example, time-varying features (e.g., volume level, proximity level, amplitude modulations, modulation depth, spectral profile, harmonicity, amplitude onsets, etc.) from the input audio signals are calculated and analyzed in real-time using a decision tree. The decision tree includes a number of hierarchical or linked branches/nodes that each perform evaluations, comparisons, or checks using at least one of the time-varying features to determine whether the set of input audio signals is “own voice” or “external voice.” In some embodiments, OVD can be conditionally used in response to a determination that the speech environment includes “speech in quiet” or “speech in noise” class (sometimes collectively referred to herein as speech classes or categories).

The conversational effort sub-category 264 indicates the user's perceived attempt or intent to process and/or provide speech-related audio signals. The conversational effort sub-category 264 can include additional sub-categories, such as a biomarker sub-category 268 and a user input sub-category 270. To this end, the conversational effort sub-category 264 can be determined via sensor data and/or user provided feedback. Indeed, due to the difficulty of determining a user's intent or attempt for conversing, different types of data can be collectively used to determine the conversational effort. The conversational effort sub-category 264 may be determined separately and independently from the conversational engagement sub-category 262. In other words, data related to the conversational engagement sub-category 262 of a user (e.g., duration of time of speaking, words used in speech) does not impact determination of the conversational effort sub-category 264. As such, the conversational effort sub-category enables determination of user participation with respect to conversations in a manner that is not indicated by the conversational engagement sub-category 262 alone. For example, a user may be in a listening situation in which the user is not able to actively speak (e.g., the user is attending a public presentation). Thus, data related to the conversational engagement sub-category 262 may initially appear to indicate reduced sociability index 250. However, the user may be actively listening (or attempting to listen) to others who are speaking, and data related to the conversational effort sub-category 264 indicates increased conversational effort. Therefore, the user's determined sociability index 250 will increase based on the conversational effort sub-category 264, even though the user is not actively speaking.

By way of example, the biomarker sub-category 268 is determined based on various biometric data related to the user and indicative of active effort made by the user to receive, process, and/or provide speech. For instance, various physiological measures (e.g., changes, values) can indicate a response or other measure of a person's internal biological state corresponding to a conversational effort. As an example, after determining that speech (e.g., non-OVD speech) is provided to the user, the user's internal biological state (e.g., emotional status, sentiment) can be determined. Such internal biological state can include, for example, a stress response (e.g., indicative of increased conversational effort), a relaxed response (e.g., indicative of maintained conversational effort), or no determined response (e.g., indicative of reduced conversational effort).

In some embodiments, the biometric data can include respiration rate (e.g., increased respiration rate indicates increased conversational effort), heart rate variability (e.g., decreased heart rate variability indicates increased conversational effort), blood pressure (e.g., increased blood pressure indicates increased conversational effort), body temperature (e.g., increased body temperature indicates increased conversational effort), and/or alpha oscillatory power (e.g., decreased alpha oscillatory power indicates increased conversational effort). The biomarker sub-category 268 can also include attributes of the own voice of the user, such as the fundamental frequency, amplitude, and/or speaking rate (e.g., increased fundamental frequency, amplitude, and/or speaking rate indicates increased conversational effort). In certain embodiments, the biometric data can be compared to baseline values, which may be determined over time (e.g., in situations in which the user does not appear to be in conversational engagement, such as in “quiet” speech environment classifications). Changes of the biometric data when non-OVD speech is detected (e.g., and when other parameters, such as a lack of substantial physical movement of the user indicated by the IMU 180, do not appear to cause biometric data changes) can indicate the conversational effort. For instance, a data model or algorithm can be used to assess the conversational effort based on various biometric data (e.g., changes in biometric data values).

The user input sub-category 270 includes a subjective rating by the user regarding the conversational effort. For example, after a detected change in the social situation (e.g., a change in the speech environment, a change in the location type), the user is prompted to provide a user input regarding the conversational effort. The user input can be provided via interaction with a user interface, such as a button, a lever, or a dial, via a gesture, such as a finger tap, a quantity of fingers held up, or head movement, and/or via audio input (e.g., a voice command). Additionally, the prompt for user input can include a free text field, a questionnaire (e.g., a Likert scale), a visual analog scale, and so forth. In some embodiments, another person, such as a significant other, a companion, or a co-worker of the user, is prompted to provide the rating of the user's conversational effort. Such a rating can indicate a more unbiased evaluation that accurately reflects the user's conversational effort.

In some embodiments, the data model or algorithm used to assess the conversational effort for the biomarker sub-category 268 can be updated based on the user input. That is, the conversational effort indicated by the user input is determined, and the data model or algorithm is updated to increase the association of corresponding biomarker data to the indicated user input. For example, during a social setting, the biometric data of the user includes an increased heart rate of 10 beats per minute and 5 breaths per minute. Additionally, the user input provided after the social setting indicates substantial conversational effort. Therefore, the data model or algorithm is updated to more strongly associate an increased heart rate of 10 beats per minute and 5 breaths per minute with substantial conversational effort. In this manner, the data model or algorithm can be adjusted based on the user input to provide a more accurate determination of conversational effort based on the biometric data.

Certain other data can further be used to determine the conversational effort. By way of example, manual adjustments to a device, such as increasing a volume of audio output resultant from operation of a hearing device, increasing a sensitivity of a microphone to receive audio signals, and/or activation of a directional microphone feature, can indicate increased conversational effort. Thus, manually changed settings of the device can be used to determine the conversational effort.

In certain examples, increased conversational effort can increase the sociability index 250, but this is not the case for all situations. For example, extremely low conversational effort may reflect disengagement from conversation, while very high conversational effort may reflect struggling in the social situation. The relationship between conversational effort and the sociability index can, in certain examples, follow a type of “stress response curve.”

The social network sub-category 266 indicates various people with whom the user converses. For example, the social network sub-category 266 can indicate whether the user is or has recently been in conversational engagement with new people and therefore has increased their social network. A frequency in which the user speaks with certain people and/or a quantity of people with whom the user speaks can be determined for the social network sub-category 266.

To this end, different people can be determined via acoustic classification based on their voice attributes (e.g., pitch, frequency, intensity, etc.) and stored in association with their voice attributes. Attributes of subsequent input audio signals are analyzed and compared to the stored voice attributes to determine whether the input audio signals are provided by a stored person. Based on a match between the attributes of the input audio signals and the stored voice attributes, a determination is made that the user is speaking with a familiar or previously identified person. However, based on there being a difference between the attributes of the input audio signals and the stored voice attributes, a determination is made that the user is in conversational engagement with a person that is different than previous people in conversational engagement with the user. That is, the user is speaking with a new person and has potentially expanded their social network. Additionally or alternatively, the social network of the user can be determined based on a communicative connection of an electronic device of the user to an electronic device of another person. For instance, expansion of the user's social network can be determined in response to determining that the mobile device of the user is communicatively coupled to (e.g., to enable call streaming) another electronic device having an unknown identity (e.g., an electronic device that has not previously been communicatively coupled to the mobile device of the user). Indeed, novel connections between the electronic device of the user and another electronic device can indicate an expanded social network. In either case, the expanded social network increases the sociability index 250.

The sociability index 250 can be determined based on any suitable combination of the aforementioned metrics. In certain embodiments, the sociability index 250 is quantitatively determined based on the metrics. For example, a numerical value (e.g., between 0 and 100) is determined for the sociability index 250. Additionally or alternatively, the sociability index 250 is qualitatively determined based on the metrics, such as to indicate a category (e.g., “high,” “low,” etc.) of the sociability index 250. In any of these cases, the different metrics can have different weight or impact to affect determination of the sociability index 250. In particular, a change in a metric having a relatively greater weight causes a greater change in the sociability index 250, whereas a change in a metric having a relatively lesser weight causes a lesser change in the sociability index 250.

In some embodiments, separate metrics for the first category 252 and for the second category 254 are independently determined, and the sociability index 250 is determined based on the separate metrics. In other words, a metric associated with the first category 252 (e.g., a speech environment metric, a call streaming metric, a location type metric) is determined regardless of the user activity, and a metric associated with the second category 254 (e.g., a conversational engagement metric, a conversational effort metric, a social network metric) is determined regardless of the associated listening situation. Indeed, the metric associated with the first category 252 and the metric associated with the second category 254 indicate different aspects of socialization for the user, and the isolated determination of the metrics can help determine a more accurate sociability index 250 by removing possible confounding or conflating variables that affect the different categories and/or sub-categories. Alternatively, certain metrics of one of the first category 252 or the second category 254 can be determined in relation with another metric of the other of the first category 252 or the second category 254. By way of example, the conversational effort metric specifically related to call streaming can be monitored, or the conversational engagement metric specifically related to speech environments classified as “speech in noise” can be monitored. Thus, more granular analysis of the user behavior can be performed to determine the sociability index 250.

A control signal can be output based on the sociability index 250. For example, the control signal can be used to notify the user of the sociability index 250, such as to provide a visual output, an audio output, or tactile feedback associated with the sociability index 250. Such a notification can prompt the user to actively manage their sociability index 250, such as to adjust or maintain certain behaviors to achieve a target level of the sociability index 250. Further actions related to the sociability index 250 can also be performed. Such actions can include sending a notification to another person (e.g., a caretaker, a significant other, a friend, a healthcare provider, etc.) to prompt the other person to assist the user, providing a recommended action to the user (e.g., behavioral changes to improve their sociability index 250), and/or adjusting a device setting (e.g., automatically enabling directional microphone capabilities to improve receipt of certain input audio signals). Thus, the control signal that is output based on the sociability index 250 can help the user with respect to different aspects of social function.

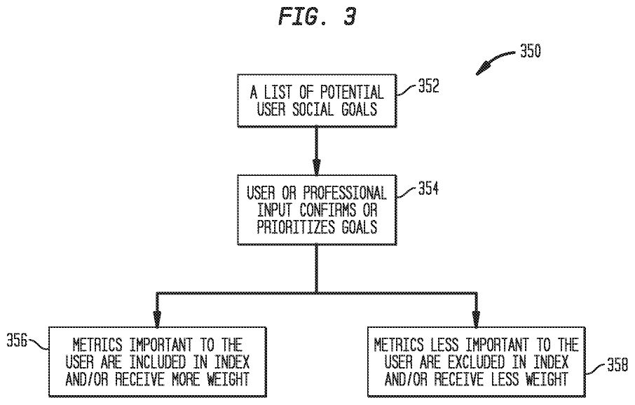

As discussed herein, different metrics can be assigned with respective weights to affect determination of the sociability index. The weights for the metrics can also be adjusted to change the determination of the sociability index. FIG. 3 is a flowchart illustrating a method 350 for adjusting weights of metrics used to determine a sociability index. For example, the weights can be automatically adjusted based on a user input. At block 352, a list of different potential user social goals are provided, such as via an electronic device of the user. The list can include various predetermined options, such as to meet new people, to connect with family, or to increase conversational contributions. The predetermined options can be associated with preset weights assigned to the different metrics. In some embodiments, multiple ones of the user social goals and/or the priority of the user social goals with respect to one another can be selected to establish the metric weights.

At block 354, the user or another person (e.g., a professional, such as an audiologist) provides an input to confirm or prioritize the user social goals. Metric weights are then adjusted accordingly. For example, at block 356, metrics regarded as relatively more important for achieving the user social goals are included and/or assigned with more weight for determination of the sociability index. At block 358, metrics regarded as relatively less important for achieving the user social goals are excluded and/or assigned with less weight for determination of the sociability index. As an example, for a user who prioritizes meeting new people as their user social goal, the social network metric of the second category 254 is included and assigned with more weight, whereas the call streaming metric of the first category 252 is excluded or assigned with less weight. As another example, for a user who prioritizes connecting with family as their user social goal, the call streaming metric of the first category 252 is included and assigned with more weight, whereas the social network metric of the second category 254 is excluded or assigned with less weight. As a further example, for a user who prioritizes increasing conversational contributions, the conversational engagement metric of the second category 254 is included and assigned with more weight, whereas the social network metric of the second category 254 is excluded or assigned with less weight. In this manner, weights for metrics of different categories and/or within the same category can be adjusted. The weight adjustment and assignment enables the determined sociability index to correspond to the user social goal more accurately and enables the user to manage their behavior in a way that is more in line with the user social goal.

It should be noted that the weights of the metrics can additionally or alternatively be adjusted or assigned based on other factors. Such factors can include a personality type of the user (e.g., the social network metric has more weight for a more extraverted user), a residence of the user (e.g., the location type metric has less weight for a user located in a more sparsely populated city), and/or device availability of the user (e.g., the call streaming metric has less weight for a user that does not own many electronic devices with call streaming capabilities). Furthermore, the weights of the metrics can be manually adjustable by the user. That is, the user can directly set the metric weights without having to provide an input that would otherwise automatically assign the weights of the metrics (e.g., to preset amounts). In addition, the goals (controlled by the weights) may be adjusted by a care provider, such as an audiologist, mental health professional or occupational therapist, etc.

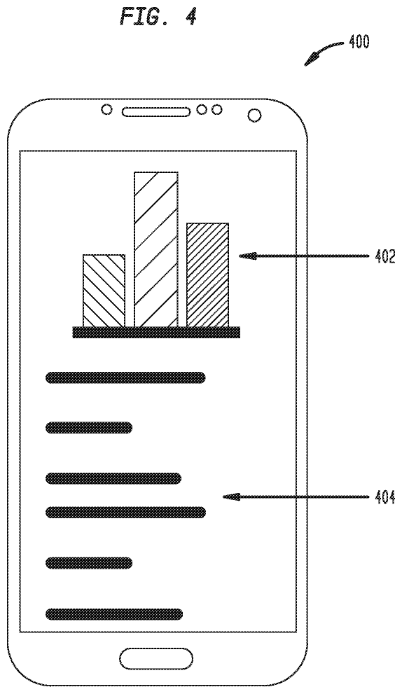

FIG. 4 is a schematic diagram illustrating a mobile device 400 (e.g., a phone, a tablet, etc.) configured to display information regarding a sociability index. For example, a control signal is output based on the determined sociability index and/or the metrics used to determine the sociability index, and the control signal instructs the mobile device 400 to display the information. The mobile device 400 includes a first display portion 402 and a second display portion 404. The first display portion 402 indicates determined sociability indexes (e.g., sociability index values) at different times. For instance, the sociability index is determined at specific times (e.g., a time of day, a day of the week) or at a particular frequency. Additionally or alternatively, the sociability index is determined with respect to certain occurrences, such as a determined social function. In either case, previously determined sociability indexes are stored and displayed at the first display portion 402. In this way, the first display portion 402 provides historical information regarding the sociability index, such as a trend of the sociability index over time. Such information can enable the user, for example, to monitor changes in social patterns and behaviors over time, such as whether their recent social patterns are affecting their sociability index in a desirable manner.

In some embodiments, information can also be used to determine a sociability index associated with the user prior to device usage (e.g., pre-implantation of a hearing device). For example, previously determined metrics, such as biomarkers and/or location data, can be transferred from another electronic device (e.g., a phone, a watch). A sociability index (e.g., a baseline sociability index) associated with the previously determined metrics can be similarly determined. As such, the user can compare a sociability index determined during device usage (e.g., post-implantation of a hearing device) with a sociability index determined prior to device usage to understand how the transition to device usage affected their social patterns.

The second display portion 404 indicates different metrics, such as any of the aforementioned metrics of the diagram 248. Thus, the second display portion 404 can inform the user of the specific metrics, such as the listening situation metrics or the activity metrics, that contribute to the determined sociability index displayed at the first display portion 402. By way of example, the second display portion 404 can provide more granular information so that the user is notified of a particular metric and can determine a more specific behavior related to the particular metric to change (e.g., increase) the sociability index.

Although the illustrated first display portion 402 and second display portion 404 include graphical representations of the sociability index and of the metrics, in additional or alternative embodiments, the sociability index and/or the metrics can be shown using a different visual representation. For instance, the mobile device 400 can display text, a table, and/or a chart. In further embodiments, instead of displaying the sociability index and the metrics via separate display portions, the mobile device 400 can display each of the sociability index and the metrics within a common display portion. In other words, a single display portion of the mobile device 400 can indicate the sociability index and the metrics. As an example, the sociability index is displayed as a bar on a bar graph, and the bar is divided into segments associated with the individual metrics contributing to the sociability index. The respective sizes of the segments correspond to the amount in which the metrics affect the sociability index (e.g., a metric, such as a metric with relatively higher weight, providing a greater contribution to the sociability index is shown as a segment with a relatively longer length).

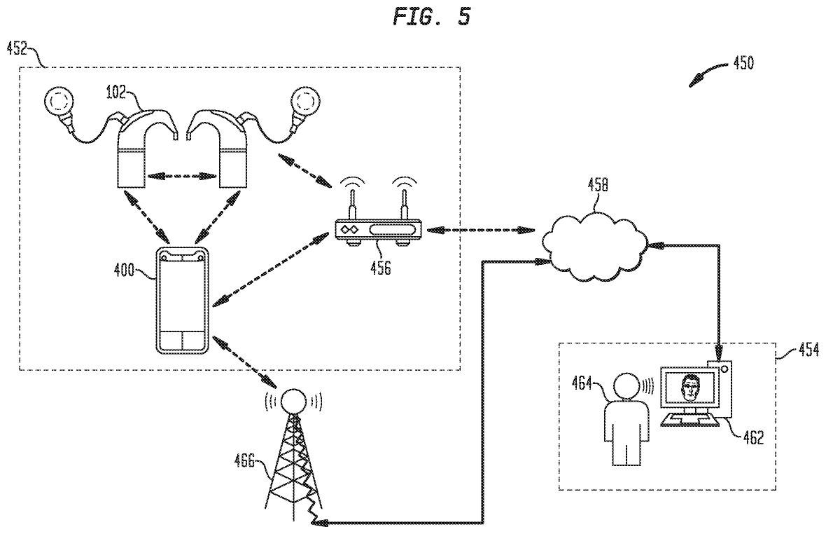

FIG. 5 is a schematic diagram illustrating a network 450 of systems that communicate information related to sociability index. In particular, such information is transmitted from user devices 452 to a professional system 454. For example, the user devices 452 include the cochlear implant system 102 and the mobile device 400 communicatively coupled to the cochlear implant system 102. The cochlear implant system 102 and the mobile device 400 can cooperatively determine various metrics and the sociability index based on the metrics. The cochlear implant system 102 and the mobile device 400 are communicatively coupled to a router 456, which is communicatively coupled to a network platform 458 (e.g., a cloud network). The network platform 458 can receive information and forward the information to certain recipients, such as to a professional device 462 (e.g., a mobile device, a desktop computer, etc.) used by a professional user 464 (e.g., a caretaker, a healthcare provider, etc.). In this way, the cochlear implant system 102 and/or the mobile device 400 can output information to the router 456, which forwards the information to the network platform 458, and the network platform 458 transmits the information to the professional device 462. Additionally or alternatively, the mobile device 400 is communicatively coupled to a terrestrial station 466 (e.g., a cell phone tower), which is communicatively coupled to the network platform 458. Thus, the mobile device 400 can transmit information to the network platform 458 via either the router 456 or the terrestrial station 466.

By way of example, the user devices 452 can transmit information to the professional device 462 to inform the professional user 464 of the sociability index of the user. Thus, the professional user 464 is also able to monitor the social patterns of the user. For instance, the professional user 464 can see whether the sociability index is at a desirable level without having to directly interact with the user. Therefore, the professional user 464 can more readily perform an action in response.

The professional user 464 can also transmit information to the user devices 452 via the network 450. As an example, the professional user 464 can utilize the professional device 462 to transmit information to the network platform 458, which then transmits the information to the router 456 and/or to the terrestrial station 466 for forwarding to the mobile device 400. For instance, the professional user 464 can manually provide a recommendation or a notification to the user to improve the sociability index. Such communication can be especially helpful to reinforce certain behaviors that may have been previously reviewed in a clinical setting but since forgotten or neglected by the user. The professional user 464 can receive information from multiple different user devices 452 that are associated with different users. Thus, the professional user 464 can monitor the social patterns of different users and provide different recommendations to the different users. Additionally, the professional user 464 can continue to monitor the sociability of a user after a recommendation (e.g., a treatment program) has been provided to the user. The professional user 464 can then determine an effectiveness of the recommendation and modify subsequent recommendations (e.g., to modify a recommended program, to avoid recommending a program) being provided.

In some embodiments, the network platform 458 can also perform operations to determine the metrics and/or the sociability index. For example, the network platform 458 can receive raw data from the mobile device 400 and/or from the cochlear implant system 102, determine the metrics based on the received raw data, and determine the sociability index based on the metrics. The network platform 458 can then transmit information related to the determined metrics and/or the determined sociability index, such as to the mobile device 400 and/or to the professional device 462. To this end, the network platform 458 includes a memory (e.g., storing instructions) and one or more processors to perform operations related to the metrics and/or the sociability index (e.g., by executing the instructions stored on the memory).

Although the network 450 illustrates various devices and systems used to communicate information between the user devices 452 and the professional system 454, it should be noted that different ways of communication can be used in additional or alternative embodiments. As an example, the mobile device 400 can be communicatively coupled to the network platform 458 via a different system, such as a non-terrestrial station (e.g., a satellite), and/or the mobile device 400 can be directly communicatively coupled to the professional device 462 (e.g., via a wired connection).

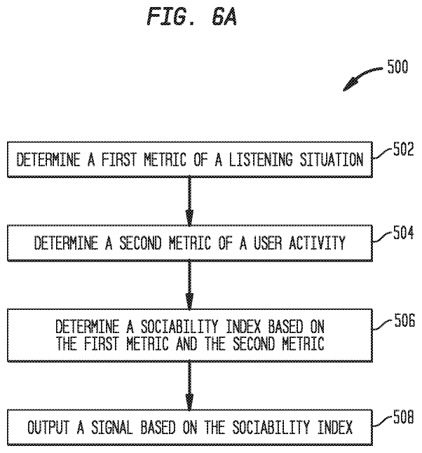

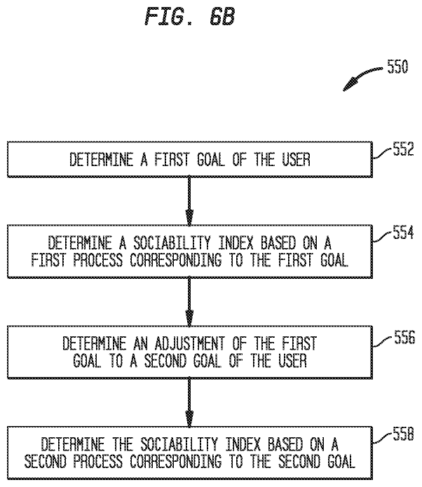

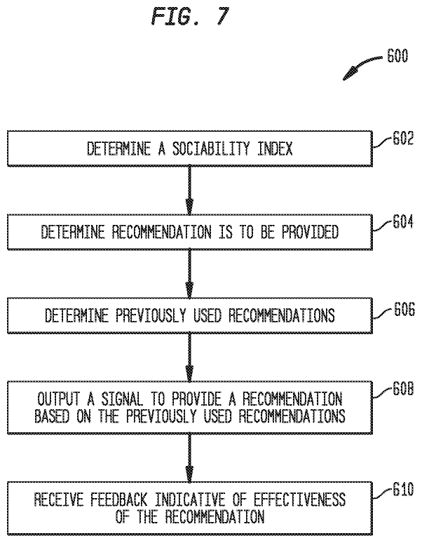

Each of FIGS. 6A, 6B, and 7 described below illustrates example methods related to sociability index operations. By way of example, the external computing device 110 of FIGS. 1A-1E can perform each of the methods. However, it should be noted that additional or alternative devices can perform the methods. Indeed, the respective methods can be performed by the same or different device. Additionally, the methods can be performed in any relation with respect to one another, such as in parallel (e.g., simultaneously) or in response to one another.

FIG. 6A is a flowchart illustrating a method 500 for determining the sociability index. At block 502, a first metric of a listening situation is determined. For example, the first metric can include a speech environment metric, a call streaming metric, and/or a location type metric. At block 504, a second metric of a user activity in the listening situation is determined. The second metric can include a conversational engagement metric, a conversational effort metric, and/or a social network metric. At block 506, a sociability index is determined based on the first metric and the second metric. For instance, an equation, an algorithm, and/or a data model is used to calculate the sociability index based on the first metric and the second metric.

At block 508, a signal (e.g., a control signal) is output based on the determined sociability index. In some embodiments, the signal causes a mobile device of the user to provide an indication or notification of the sociability index. For instance, a visual output (e.g., a display), an audio output (e.g., an alarm), and/or tactile feedback (e.g., a vibration) can be provided to the user. By informing the user of the sociability index, the user can be prompted to improve and/or maintain their sociability index, such as to set and achieve a target sociability index. The signal can cause an electronic device of another user, such as a professional, to provide an indication or notification of the sociability index to the other user. The other user can then monitor progress of the user and perform a corresponding action, such as to incorporate a potential program for the user to improve the sociability index. In additional or alternative embodiments, the signal can adjust certain device settings. By way of example, for a low sociability index, the signal can change signal processing related operations of the device and/or enable microphone directionality to facilitate conversational effort and increase the sociability index. In further embodiments, the signal can be output to provide a recommendation to the user. For example, the recommendation can include a suggested action that the user can perform, such as to turn down background noise (e.g., music), to improve their sociability index in certain situations. The recommendation can also include suggested device setting adjustments for the user to manually change the device settings to improve their sociability index. Further still, the signal can help the user achieve a desirable sociability index in other ways. As an example, the signal can provide the user with an incentive to maintain or achieve a target sociability index, such as by crediting the user with financial benefits (e.g., discounts, credits, gift cards, etc.) for certain items or stores. This gamification of the sociability index can further encourage the user to actively manage their sociability index. In certain embodiments, the signal is output based on the sociability index as compared to a target or threshold level, such as in response to the sociability index being less than the target level (e.g., the sociability index is to be improved) or greater than the target level (e.g., the sociability index is to be maintained).

As discussed herein, the manner in which the sociability index is determined can be adjusted to be more customized or personalized. FIG. 6B is a flowchart illustrating a method 550 for determining the sociability index. At block 552, a first goal of the user is determined, such as based on a user input (e.g., by the user or by a professional). At block 554, the sociability index is determined based on a first process (e.g., an algorithm, an equation, a data model, etc.) corresponding to the first goal. For example, the sociability index is determined based on various metrics, and each metric can be weighted differently. Certain metrics can be more important for achievement of the first goal, so the first process includes a first set of weights assigned to the metrics in which metrics that are more important are assigned with relatively higher weights. As such, the sociability index determined via the first process more closely corresponds to the first goal.

At block 556, an adjustment of the first goal to a second goal is determined. At block 558, the sociability index is determined based on a second process corresponding to the second goal. For instance, a different metric can be more important for achievement of the second goal as compared to achievement of the first goal. Therefore, the second process can include a second set of weights, different from the first set of weights, assigned to the metrics such that the sociability index determined via the second process more closely corresponds to the second goal.

It should be noted that determination of the sociability index can further be adjusted based on other factors, such as a personality of the user. For example, the sociability index can be determined in different manners for extraverted and introverted users. Indeed, because different metrics can be of a different importance for users of different personalities, determination of the sociability index can be adjusted to more closely represent a particular personality, such as by changing the metric weights such that metrics more important for a personality are assigned with greater weights. In this manner, the sociability index can more closely reflect the user and enable the user to manage the sociability index more effectively.

FIG. 7 is a flowchart illustrating a method 600 for providing a recommendation based on a sociability index. At block 602, a sociability index is determined based on various metrics. At block 604, a determination is made that a recommendation is to be provided based on the sociability index. For example, the recommendation is to be provided in response to the sociability index being below a target level, so the recommendation can encourage the user to improve their sociability index.

At block 606, previously used recommendations are determined. By way of example, recommendations that were previously provided to the user are stored and retrieved. Additionally or alternatively, recommendations that were previously provided to other users are stored and retrieved. For instance, information related to multiple users is accessed (e.g., from a cloud server), and a recommendation is identified based on such information. At block 608, a signal is output to cause an electronic device (e.g., a mobile device) of the user to provide a recommendation based on the previously used recommendations, such as by selecting the recommendation from a list of the previously used recommendations. Such a recommendation can be provided via a visual output, an audio output, tactile feedback, and/or any other suitable notification.

In some embodiments, the specific recommendation to be provided is based on the metrics used to determine the sociability index. Thus, the recommendation can be tailored to the user's particular social context. In an example, based on a highly weighted conversational engagement metric being undesirable, the recommendation can recommend actions to improve the conversational engagement metric, including reducing background music volume, orienting to face away from noise sources, activating directional microphone features, moving closer to a speaker, asking a speaker to reduce their speaking rate, and the like. In another example, the recommendation can be based on different detected noise sources. Indeed, the context of the user can include different sources of noise distortion, such as steady-state noise (e.g., surrounding machinery in operation), a quantity of speakers (e.g., competing speakers), reverberation or echo of noise, background music, or impulse noise (e.g., dish clatter), that can reduce the user's ability to hear (e.g., to reduce the conversational effort metric and/or the conversational engagement metric). Thus, the recommendation can specifically include mitigation of such noise sources. Therefore, the recommendation provided to the user can be tailored to a particularly determined context to provide customized actional support that can be effectively implemented to improve the sociability index.

At block 610, feedback indicative of the effectiveness of a provided recommendation is received. The feedback can be provided by the user or by a different person (e.g., a professional, a significant other, etc.) to indicate the effectiveness of the recommendation. For example, the feedback can indicate that the recommendation effectively improved a metric (e.g., facilitated the conversational effort metric) or did not improve the metric. The feedback can then be used for providing subsequent recommendations. By way of example, based on the feedback indicating that the recommendation was effective, the recommendation can be provided more frequently in the future. However, based on the feedback indicating that the recommendation was not effective, the recommendation may not be provided or may be provided less frequently in the future.

A recommendation can also be provided based on a trend of the sociability index. For example, the signal is output to provide a recommendation (e.g., to change a certain behavior) based on the sociability index decreasing at above a threshold rate. A recommendation can also be provided even when the sociability index is desirable (e.g., above a threshold level). As an example, the recommendation can include suggestions to maintain the desirable sociability index and to reinforce certain social patterns. Further still, a recommendation can be provided based on metrics regardless of the sociability index. For instance, a signal can be provided to improve the conversational engagement metric (e.g., to activate a directional microphone) even though the sociability index is desirable (e.g., because the conversational effort metric is high). As such, the recommendation being provided can improve social patterns of the user in multiple different contexts.

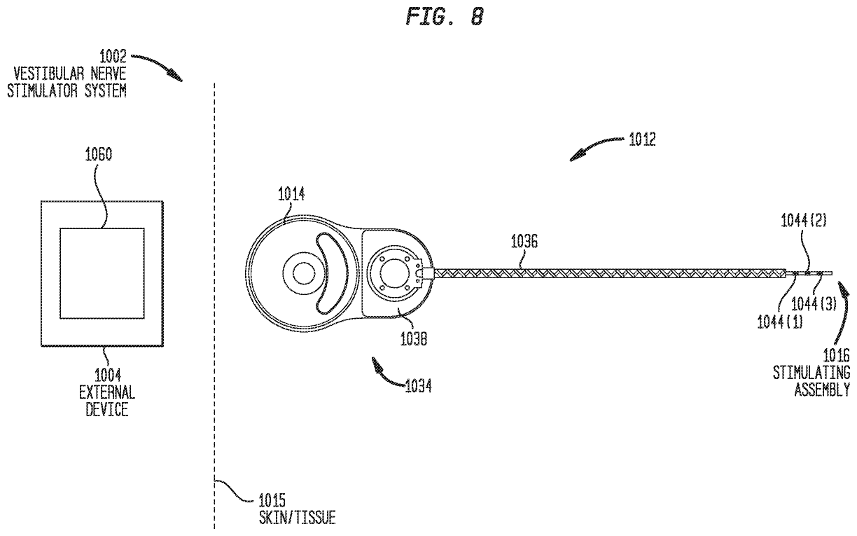

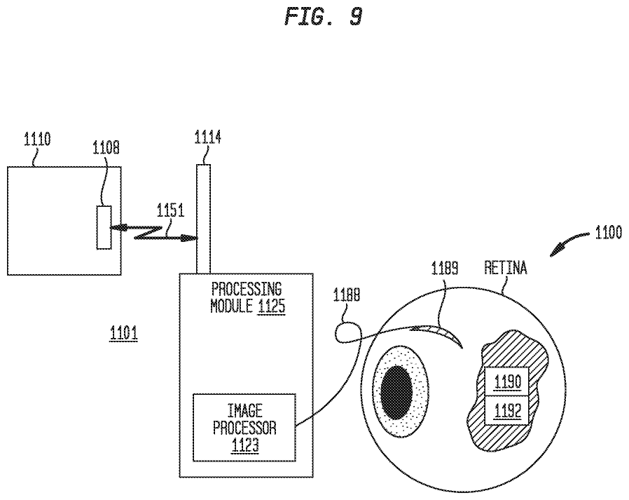

As previously described, the technology disclosed herein can be applied in any of a variety of circumstances and with a variety of different devices. Example devices that can benefit from technology disclosed herein are described in more detail in FIGS. 8 and 9. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 8 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012.

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1012, the external device 1004, and/or another external device can be configured to implement the techniques presented herein. That is, the vestibular stimulator 1012, possibly in combination with the external device 1004 and/or another external device, can include an evoked biological response analysis system, as described elsewhere herein.

FIG. 9 illustrates a retinal prosthesis system 1101 that comprises an external device 1110 configured to communicate with an implantable retinal prosthesis 1100 via signals 1151. The retinal prosthesis 1100 comprises an implanted processing module 1125, and a retinal prosthesis sensor-stimulator 1190 is positioned proximate the retina of a recipient. The external device 1110 and the processing module 1125 can communicate via coils 1108, 1114.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that converts the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190 and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire, and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

The technology described and claimed herein is not to be limited in scope by the specific preferred embodiments herein disclosed, since these embodiments are intended as illustrations, and not limitations, of several aspects of the disclosure. Any equivalent embodiments are intended to be within the scope of this disclosure. Indeed, various modifications of the disclosure in addition to those shown and described herein will become apparent to those skilled in the art from the foregoing description. Such modifications are also intended to fall within the scope of the appended claims.

Additionally, when used herein, the term “comprises” and its derivations (such as “comprising”) should not be understood in an excluding sense, that is, these terms should not be interpreted as excluding the possibility that what is described and defined can include further elements, steps, etc. Similarly, where any description recites “a” or “a first” element or the equivalent thereof, such disclosure should be understood to include incorporation of one or more such elements, neither requiring nor excluding two or more such elements. Meanwhile, when used herein, the term “approximately” and terms of its family (such as “approximate”) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies. For example, the term “approximately” can denote a tolerance of plus or minus 0.002 inches, 0.001 inches, or up to 0.005 inches. The same applies to the terms “about” and “around” and “substantially.” Moreover, for the purposes of the present disclosure, the phrase “A and/or B” means (A), (B), or (A and B), and the phrase “A, B, and/or C” means (A), (B), (C), (A and B), (A and C), (B and C), or (A, B and C).

Finally, the techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [performing]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method, comprising:
receiving, via a processor, biometric data of a hearing device user;
determining, via the processor, a sociability index based on the biometric data; and
outputting, via the processor, a signal based on the sociability index.

2. The method of claim 1, wherein outputting the signal based on the sociability index comprises:
outputting, via the processor, a notification in response to determining a difference between the sociability index and a target level exceeds a threshold value.

3. The method of claim 1, comprising:
determining, via the processor, a location of the hearing device user; and
determining, via the processor, the sociability index based on the biometric data and the location.

4. The method of claim 3, wherein determining the sociability index based on the biometric data and the location comprises:
comparing, via the processor, the location with a set of previous locations of the hearing device user; and
determining, via the processor, the sociability index based on a difference between the location and the set of previous locations.

5. The method of claim 1, wherein determining the sociability index based on the biometric data comprises:
receiving, via the processor, non-own voice detection (non-OVD) speech; and
determining, via the processor, the sociability index based on the biometric data received upon receipt of the non-OVD speech.

6. The method of claim 5, wherein determining the sociability index based on the biometric data received upon receipt of the non-OVD speech comprises:
determining, via the processor, an internal biological state of the hearing device user upon receipt of the non-OVD speech; and
determining, via the processor, the sociability index based on the internal biological state.

7. The method of claim 5, wherein determining the sociability index based on the biometric data received upon receipt of the non-OVD speech comprises:
receiving, via the processor, first biometric data prior to receipt of the non-OVD speech;
receiving, via the processor, second biometric data after receipt of the non-OVD speech;
determining, via the processor, a difference between the first biometric data and the second biometric data; and
determining, via the processor, the sociability index based on the difference between the first biometric data and the second biometric data.

8. One or more non-transitory computer readable storage media comprising instructions that, when executed by a processor, cause the processor to:
receive biometric data from a sensor associated with a device configured to be worn by a user;
determine, based on the biometric data, a conversational effort of the user; and
determine a sociability index based on the conversational effort.

9. The one or more non-transitory computer readable storage media of claim 8, wherein the instructions, when executed by the processor, cause the processor to:
receive a first user input indicative of a first goal of the user;
determine the sociability index based on the conversational effort of the user in correspondence to the first goal;
determine an adjustment of the first goal to a second goal of the user; and
determine an adjusted sociability index based on the conversational effort of the user in correspondence to the second goal.

10. The one or more non-transitory computer readable storage media of claim 9, wherein the instructions, when executed by the processor, cause the processor to determine the sociability index based on the conversational effort of the user in correspondence to the first goal by applying a first weighted value to the conversational effort of the user.

11. The one or more non-transitory computer readable storage media of claim 10, wherein the instructions, when executed by the processor, cause the processor to determine the adjusted sociability index based on the conversational effort of the user in correspondence to the second goal by applying a second weighted value, different from the first weighted value, to the conversational effort of the user.

12. The one or more non-transitory computer readable storage media of claim 8, wherein the instructions, when executed by the processor, cause the processor to:
output a signal based on the sociability index.

13. The one or more non-transitory computer readable storage media of claim 12, wherein the instructions, when executed by the processor, cause the processor to:
output the signal to cause a mobile device of the user to display the sociability index.

14. The one or more non-transitory computer readable storage media of claim 12, wherein the instructions, when executed by the processor, cause the processor to:
output the signal to adjust a setting of the device configured to be worn by the user.

15. The one or more non-transitory computer readable storage media of claim 8, wherein the instructions, when executed by the processor, cause the processor to:
compare the biometric data with baseline biometric data associated with the user; and
determine the conversational effort of the user based on a difference between the biometric data and the baseline biometric data.

16. The one or more non-transitory computer readable storage media of claim 15, wherein the instructions, when executed by the processor, cause the processor to:
receive initial biometric data from the sensor over a duration of time; and
determine the baseline biometric data based on the initial biometric data received from the sensor over the duration of time.

17-28. (canceled)

29. A device, comprising:
a memory configured to store instructions; and
one or more processors that, upon executing the instructions stored on the memory, are configured to:
determine a first metric of a listening situation associated with a user of the device;
determine a second metric of biometric data of the user in the listening situation; and
determine a sociability index based on the first metric and the second metric.

30. The device of claim 29, wherein the first metric of the listening situation comprises a quantity or duration of time of call streaming.

31. (canceled)

32. The device of claim 29, wherein the device is a hearing device, and the one or more processors, upon executing the instructions stored on the memory, are configured to generate output signals for delivery to the user.

33. (canceled)

34. The device of claim 29, wherein the second metric of biometric data of the user in the listening situation comprises:
an internal biological state of the user in the listening situation.

35. (canceled)

36. (canceled)
